(19) United States
(12) Patent Application Publication
(10) Pub. No.: US 2025/0205449 A1
(43) Pub. Date: Jun. 26, 2025

(54) SPACE PRESENTATION APPARATUS, SPACE PRESENTATION METHOD, AND NON-TRANSITORY COMPUTER-READABLE MEDIUM

(71) Applicant: Yokogawa Electric Corporation, Tokyo (JP)

(72) Inventors: Kiyomi MURAOKA, Tokyo (JP); Motohiko ISHIDA, Tokyo (JP); Hirotaka YAMASHITA, Tokyo (JP); Takashi TSUBOTA, Tokyo (JP)

(21) Appl. No.: 18/916,664

(22) Filed: Oct. 15, 2024

(30) Foreign Application Priority Data
Dec. 26, 2023 (JP) 2023-219210

(51) Int. Cl. A61M 21/00; A61B 5/16

(52) U.S. Cl. CPC A61M 21/00; A61B 5/165; A61M 2021/005; A61M 2205/50; A61M 2205/583; A61M 2230/005; A61M 2230/10

(57) ABSTRACT

A space presentation apparatus comprising an information acquisition unit which acquires subject brain wave information which is brain wave information of a subject, and a space presentation unit which presents a representation space to the subject is provided. The information acquisition unit acquires first brain wave information which is the subject brain wave information before the representation space is presented. The space presentation unit presents the representation space depending on the first brain wave information. The information acquisition unit acquires second brain wave information which is the subject brain wave information after the representation space is presented. The space presentation unit adjusts the representation space based on a change from the first brain wave information to the second brain wave information. The space presentation apparatus may further include a state estimation unit which estimates a state of the subject based on the subject brain wave information of the subject.

The contents of the following patent application(s) are incorporated herein by reference: NO. 2023-219210 filed in JP on Dec. 26, 2023

### BACKGROUND

### 1. Technical Field

The present invention relates to a space presentation apparatus, a space presentation method, and a non-transitory computer-readable medium.

### 2. Related Art

Patent Document 1 describes in the abstract, “information on a scent which is suitable for all of a plurality of users is provided, based on mental states of all of the plurality of users in a predetermined space.”

Patent Document 2 describes in the abstract, “Such systems are configured to monitor the brain state of an individual and configured to create a feedback loop whereby the brain state of the individual modulates parameters of the augmented reality system”.

### PRIOR ART DOCUMENTS

### Patent Documents

Patent Document 1: Japanese Patent Application Publication No. 2021-108918

Patent Document 2: Japanese Translation Publication of a PCT Route Patent Application No. 2021-511612

### BRIEF DESCRIPTION OF THE DRAWINGS

FIG. 1 is a diagram illustrating an example of a subject 110 before a representation space 200 is presented.

FIG. 2 is a diagram illustrating an example of the subject 110 after the representation space 200 is presented.

FIG. 3 is a diagram illustrating another example of the subject 110 before the representation space 200 is presented.

FIG. 4 is a diagram illustrating an example of the subject 110 after the representation space 200 is presented.

FIG. 5 is a block diagram illustrating an example of a space presentation apparatus 100 according to an embodiment of the present invention.

FIG. 6 is a diagram illustrating an example of an information acquisition unit 10.

FIG. 7 is a diagram illustrating an example of a state S estimated by a state estimation unit 30.

FIG. 8 is a diagram illustrating an example of line-of-sight information of the subject 110.

FIG. 9 is a diagram illustrating an example of a relationship between presentation time of the representation space 200 and a state of a brain wave of the subject 110.

FIG. 10 is a flowchart illustrating an example of a space presentation method according to an embodiment of the present invention.

FIG. 11 is a diagram illustrating an example of a computer 2200 in which the space presentation apparatus 100 according to an embodiment of the present invention may be embodied entirely or partially.

### DESCRIPTION OF EXEMPLARY EMBODIMENTS

Hereinafter, while the present invention will be described through the embodiments of the invention, the following embodiments do not limit the invention according to the claims. In addition, not all combinations of features described in the embodiments are essential to the solution of the invention.

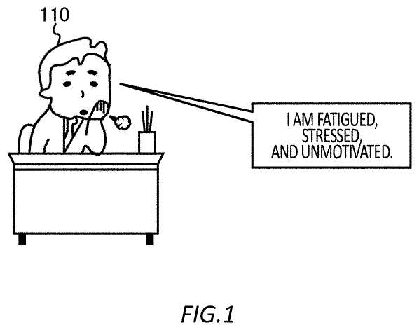

FIG. 1 is a diagram illustrating an example of a subject 110 before a representation space 200 described below is presented. In the example of FIG. 1, the subject 110 is sitting at his/her desk in an office. A state of the subject 110 is defined as a state S. The state S may be a conscious state or a subconscious state of the subject 110. The conscious state of the subject 110 is a psychological state of the subject 110 which is recognized by the subject 110 on his/her own. The subconscious state of the subject 110 is a psychological state of the subject 110 which is not recognized by the subject 110 on his/her own. In the example of FIG. 1, before the representation space 200 described below is presented, the subject 110 feels “I am fatigued, stressed, and unmotivated”.

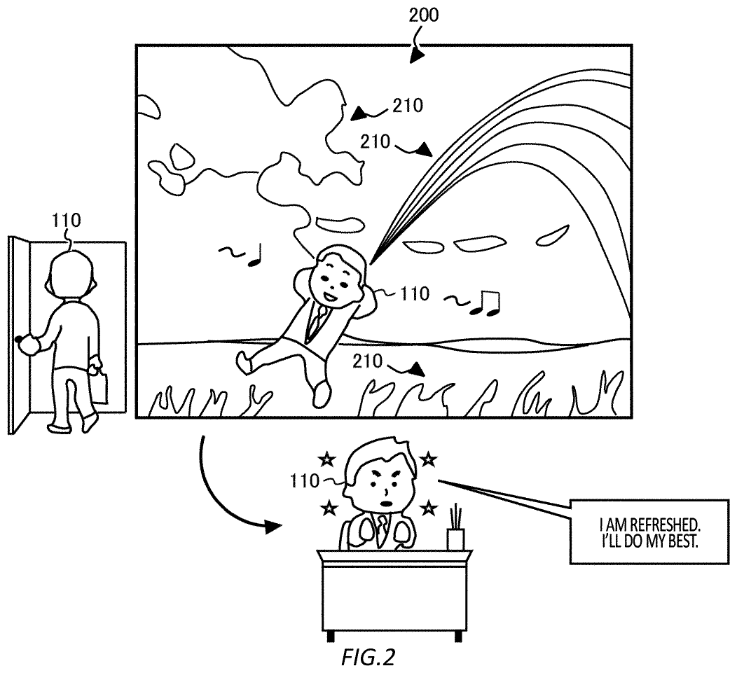

FIG. 2 is a diagram illustrating an example of the subject 110 after the representation space 200 is presented. “After the representation space 200 is presented” may refer to after the presentation of the representation space 200 is started, during the presentation of the representation space 200, or after the presentation of the representation space 200 is finished. In the example of FIG. 2, the subject 110 stands up from his/her desk in the example of FIG. 1 and moves to another room where the representation space 200 is presented. In the example of FIG. 2, the representation space 200 is presented to the subject 110 in the another room.

The representation space 200 may be a virtual space or a space where image elements 210 are displayed in a real space. The image elements 210 may be virtual objects. The image elements 210 being displayed in the real space may refer to the image elements 210 being displayed on a wall, a ceiling, or the like in the real space, or may refer to the image elements 210 in a form of video being displayed on the wall, the ceiling or the like in the real space. In the example of FIG. 2, the image elements 210 include a grassland, a cloud, and a rainbow. In the example of FIG. 2, the image elements 210 are displayed in a superimposed manner in the real space.

In the example of FIG. 2, the subject 110 returns to his/her desk in the example of FIG. 1 from the another room where the representation space 200 is presented. In the example of FIG. 2, after the representation space 200 is presented, the subject 110 feels “I am refreshed. I′ll do my best”.

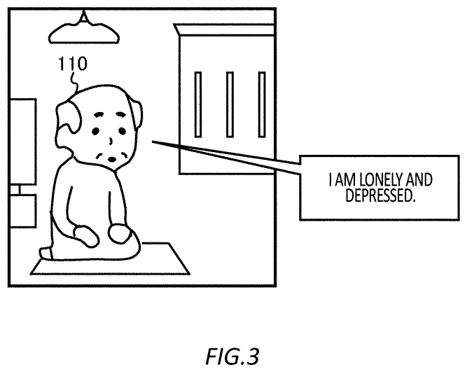

FIG. 3 is a diagram illustrating another example of the subject 110 before the representation space 200 is presented. In the example of FIG. 3, the subject 110 is sitting on a floor in his/her house. In the example of FIG. 3, before the representation space 200 is presented, the subject 110 feels “I am lonely and depressed”.

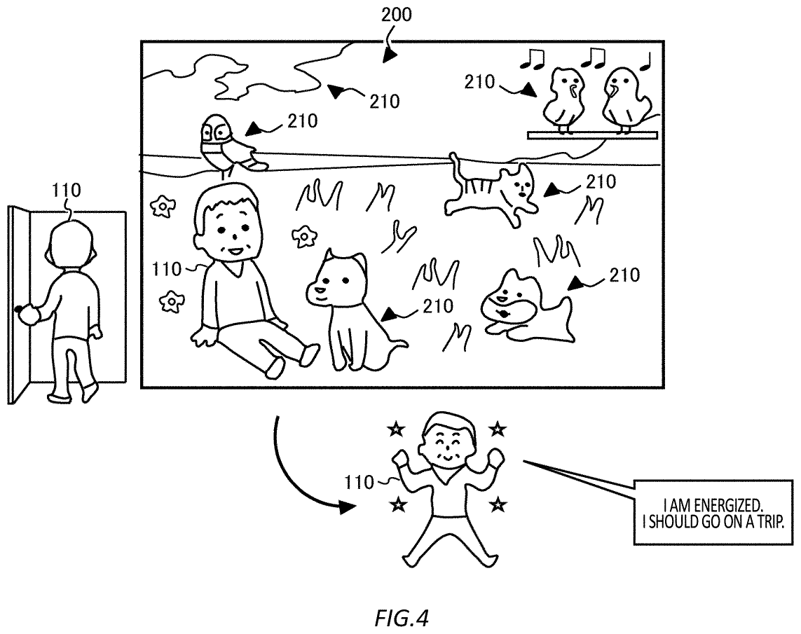

FIG. 4 is a diagram illustrating an example of the subject 110 after the representation space 200 is presented. In the example of FIG. 4, the subject 110 stands up from the state of sitting on the floor in the example of FIG. 3 and moves to another room where the representation space 200 is presented. The representation space 200 is presented to the subject 110 in the another room. In the example of FIG. 4, the image elements 210 include an animal such as a dog and a bird, a grassland, and a cloud. In the example of FIG. 4, the image elements 210 are displayed in the real space. In the example of FIG. 4, after the representation space 200 is presented, the subject 110 feels “I am energized. I should go on a trip.”

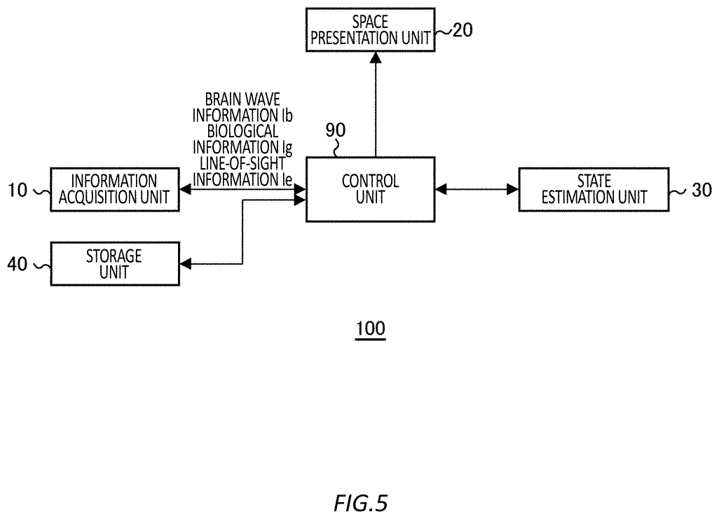

FIG. 5 is a block diagram illustrating an example of a space presentation apparatus 100 according to an embodiment of the present invention. The space presentation apparatus 100 includes an information acquisition unit 10 and a space presentation unit 20. The space presentation apparatus 100 may include a state estimation unit 30, a storage unit 40, and a control unit 90.

The space presentation apparatus 100 may be realized by a computer partially or entirely. The control unit 90 may be a central processing unit (CPU) of the computer. When the space presentation apparatus 100 is realized by a computer, a space presentation program which causes the computer to function as the space presentation apparatus 100, or a space presentation program which causes the computer to execute a space presentation method described below may be installed on the computer.

Brain wave information of the subject 110 is defined as subject brain wave information Ib. The information acquisition unit 10 acquires the subject brain wave information Ib of the subject 110. The subject brain wave information Ib may be information for reproducing at least a part of a temporal waveform of a brain wave of the subject 110. The subject brain wave information Ib may include data obtained by sampling the temporal waveform of the brain wave, or may include data indicating a magnitude of a frequency component of a brain wave at one or more frequencies, or may include another data. For example, the subject brain wave information Ib includes data indicating a magnitude of a component of at least one of a delta wave, which is less than 4 Hz, a theta wave, which is 4 Hz or more and less than 8 Hz, an alpha wave, which is 8 Hz or more and less than 14 Hz, a beta wave, which is 14 Hz or more and less than 26 Hz, or a gamma wave, which is 26 Hz or more and less than 40 Hz.

The alpha wave may be further classified into a low alpha wave, which is 8 Hz or more and less than 10 Hz, a medium alpha wave, which is 10 Hz or more and less than 12 Hz, and a high alpha wave, which is 12 Hz or more and less than 14 Hz, depending on a frequency band. The subject brain wave information Ib may include data indicating a magnitude of at least one of the low alpha wave, the medium alpha wave, or the high alpha wave.

The beta wave may be further classified into a low beta wave, which is 14 Hz or more and less than 18 Hz, and a high beta wave, which is 18 or more and less than 26 Hz, depending on a frequency band. The subject brain wave information Ib may include data indicating a magnitude of at least one of the low beta wave or the high beta wave.

The subject brain wave information Ib may include information of the temporal waveforms of one or more brain waves measured at one or more positions in a head part including a head and a face of the subject 110. For example, the subject brain wave information Ib may be acquired by measuring the temporal waveforms of potentials at electrodes arranged equidistantly in a vicinity of a scalp of the subject 110, as in the international 10-20 system, or may be acquired by another method. A plurality of electrodes arranged on the scalp may not be equidistant from each other. The electrodes may be provided in a wearable appliance to be worn on the head part of the subject 110, such as headgear, headphones, earphones, and glasses. The subject brain wave information Ib may be information obtained by acquiring an electrical signal at an electrode implanted in a body of the subject 110 via wireless communication.

A sum of amplitudes of the alpha wave, the beta wave, the theta wave, the gamma wave, and the delta wave at a certain timing is defined as a total amplitude As. As an example, when a proportion of the amplitude of the delta wave of the subject 110 to the total amplitude As is greater than any of a proportion of the amplitude of the alpha wave to the total amplitude As, a proportion of the amplitude of the beta wave to the total amplitude As, a proportion of the amplitude of the theta wave to the total amplitude As, and a proportion of the amplitude of the gamma wave to the total amplitude As, it may be assumed that the subject 110 is in a sleeping state.

As an example, when the proportion of the amplitude of the theta wave of the subject 110 to the total amplitude As is increasing over time, it may be assumed that fatigue and drowsiness of the subject 110 are increasing. As an example, when a proportion of a sum of an amplitude of the low alpha wave and an amplitude of the medium alpha wave of the subject 110 to the total amplitude As is increasing over time, it may be assumed that a degree of relaxation of the subject 110 is increasing. As an example, when a proportion of a sum of an amplitude of the high alpha wave and an amplitude of the low beta wave of the subject 110 to the total amplitude As is increasing over time, it may be assumed that a state of the subject 110 in which relaxation and concentration are well-balanced is increasing. The state in which the relaxation and the concentration are well-balanced is a so-called immersed state.

The subject brain wave information Ib before the representation space 200 is presented is defined as first brain wave information Ib1. The information acquisition unit 10 acquires the first brain wave information Ib1. The space presentation unit 20 presents the representation space 200 to the subject 110. The space presentation unit 20 presents the representation space 200 depending on the first brain wave information Ib1. The representation space 200 may include image elements 210. The representation space 200 depending on the first brain wave information Ib1 is a space which may put the state S described below of the subject 110 into a desired state S described below of the subject 110.

When the proportion of the amplitude of the theta wave to the total amplitude As in the first brain wave information Ib1 is increasing over time, the representation space 200 depending on the first brain wave information Ib1 may refer to the representation space 200 for relieving the fatigue and the drowsiness of the subject 110. When the proportion of the sum of the amplitude of the low alpha wave and the amplitude of the medium alpha wave to the total amplitude As in the first brain wave information Ib1 is increasing over time, the representation space 200 depending on the first brain wave information Ib1 may refer to the representation space 200 for enhancing the degree of relaxation of the subject 110. When the proportion of the sum of the amplitude of the high alpha wave and the amplitude of the low beta wave to the total amplitude As in the first brain wave information Ib1 is increasing over time, the representation space 200 depending on the first brain wave information Ib1 may refer to the representation space 200 for enhancing a degree of immersion of the subject 110.

The subject brain wave information Ib after the representation space 200 is presented is defined as second brain wave information Ib2. The information acquisition unit 10 acquires the second brain wave information Ib2. The space presentation unit 20 adjusts the representation space 200 based on a change from the first brain wave information Ib1 to the second brain wave information Ib2. For example, when the proportion of the amplitude of the theta wave to the total amplitude As in the second brain wave information Ib2 is increased from the proportion of the amplitude of the theta wave to the total amplitude As in the first brain wave information Ib1, though the representation space 200 depending on the first brain wave information Ib1 is the representation space 200 for relieving the fatigue and the drowsiness of the subject 110, there is a high probability that the fatigue and the drowsiness of the subject 110 is not relieved by the presented representation space 200. In such a case, the space presentation unit 20 adjusts the representation space 200. Adjusting the representation space 200 may refer to adjusting brightness of the representation space 200, or may refer to changing a type K described below of the representation space 200. When scenery such as a sea and a mountain is presented in the representation space 200 depending on the first brain wave information Ib1, for example, changing the type K described below of the representation space 200 may refer to presenting an animal such as a dog and a cat instead of the scenery. Adjusting the representation space 200 may refer to presenting music in the representation space 200.

The type K of the representation space 200 refers to a type of the representation space 200 which may put the subject 110 into the desired state S. The type K and the state S may be associated with each other in advance. For example, when a type K is scenery such as a sea and a mountain, the state S in which the subject 110 feels calm may be associated with the type K, when a type K is an animal such as a dog and a cat, the state S in which the subject 110 feels calm may be associated with the type K, when a type K is a jungle in an adventure of unexplored land, the state S in which the subject 110 feels active and immersed may be associated with the type K. The type K and the state S associated with each other may be stored in the storage unit 40.

For example, when the representation space 200 depending on the first brain wave information Ib1 is the representation space 200 for enhancing the degree of relaxation of the subject 110, and the proportion of the sum of the amplitude of the low alpha wave and the amplitude of the medium alpha wave to the total amplitude As in the second brain wave information Ib2 is increased from the proportion of the sum of the amplitude of the low alpha wave and the amplitude of the medium alpha wave to the total amplitude As in the first brain wave information Ib1, there is a high probability that the degree of relaxation of the subject 110 is enhanced by the presented representation space 200. In such a case, the space presentation unit 20 may adjust the representation space 200 to further enhance the degree of relaxation. In the present example, adjusting the representation space 200 refers to adding a representation to the representation space 200 without changing the type K of the representation space 200. Adding a representation may refer to, for example, adding image elements 210 such as a dog and a cat to the representation space 200.

For example, when the representation space 200 depending on the first brain wave information Ib1 is the representation space 200 for enhancing the degree of immersion of the subject 110, and the proportion of the sum of the amplitude of the high alpha wave and the amplitude of the low beta wave to the total amplitude As in the second brain wave information Ib2 is increased from the proportion of the sum of the amplitude of the high alpha wave and the amplitude of the low beta wave to the total amplitude As in the first brain wave information Ib1, there is a high probability that the degree of immersion of the subject 110 is enhanced by the presented representation space 200. In such a case, the space presentation unit 20 may adjust the representation space 200 to enhance the degree of immersion. In the present example, adjusting the representation space 200 refers to adding a representation to the representation space 200 without changing the type K of the representation space 200. Adding a representation may refer to, for example, presenting music for further enhancing the degree of immersion.

The subject brain wave information Ib may reflect the conscious state S or the subconscious state S of the subject 110. The subconscious state S may reflect the psychological state of the subject 110 which is not recognized by the subject 110 on his/her own. The space presentation unit 20 adjusts the representation space 200 based on a change from the first brain wave information Ib1 to the second brain wave information Ib2. Accordingly, the subject 110 is more likely to become the desired state S.

The state estimation unit 30 may estimate a state of the subject 110 based on the subject brain wave information Ib of the subject 110. The state is defined as a state S. The state estimation unit 30 may estimate the state S of the subject 110 based on the change from the first brain wave information Ib1 to the second brain wave information Ib2. The space presentation unit 20 may present the representation space 200 or may adjust the representation space 200 based on the state S of the subject 110.

As an example, when the proportion of the amplitude of the theta wave to the total amplitude As in the second brain wave information Ib2 is greater than the proportion of the amplitude of the theta wave to the total amplitude As in the first brain wave information Ib1, the state estimation unit 30 may estimate that the state S of the subject 110 is a state in which the fatigue and the drowsiness are increasing. The space presentation unit 20 may present the representation space 200 for relieving the state S in which the fatigue and the drowsiness are increasing.

As an example, when the proportion of the sum of the amplitude of the low alpha wave and the amplitude of the medium alpha wave to the total amplitude As in the second brain wave information Ib2 is greater than the proportion of the sum of the amplitude of the low alpha wave and the amplitude of the medium alpha wave to the total amplitude As in the first brain wave information Ib1, the state estimation unit 30 may estimate that the state S of the subject 110 is a state in which the degree of relaxation is increasing. The space presentation unit 20 may present the representation space 200 for enhancing the state S in which the degree of relaxation is increasing.

As an example, when the proportion of the sum of the amplitude of the high alpha wave and the amplitude of the low beta wave to the total amplitude As in the second brain wave information Ib2 is greater than the proportion of the sum of the amplitude of the high alpha wave and the amplitude of the low beta wave to the total amplitude As in the first brain wave information Ib1, the state estimation unit 30 may estimate that the state S of the subject 110 is a state in which the relaxation and the concentration are well-balanced. The state in which the relaxation and the concentration are well-balanced is a so-called immersed state. The space presentation unit 20 may present the representation space 200 for enhancing the state S in which the degree of immersion is increasing.

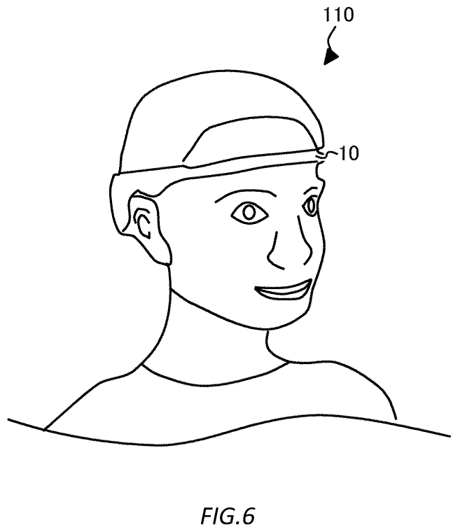

FIG. 6 is a diagram illustrating an example of the information acquisition unit 10. The information acquisition unit 10 may include an electroencephalograph which may measure the subject brain wave information Ib, or may include communication equipment which acquires the subject brain wave information Ib measured by an external electroencephalograph. The information acquisition unit 10 of the present example is an electroencephalograph in a form of headgear. The information acquisition unit 10 may be an electroencephalograph in a form of earphones. In the present example, the representation space 200 may be presented to the subject 110 with an electroencephalograph in a form of headgear or earphones being worn.

When the information acquisition unit 10 is an electroencephalograph in a form of headgear, the space presentation unit 20 and the control unit 90 may not be accommodated in a housing of the headgear. The subject brain wave information Ib acquired by the information acquisition unit 10 may be transmitted to the control unit 90 via a wireless connection.

The information acquisition unit 10 may further acquire biological information of the subject 110. The biological information is defined as biological information Ig. The information acquisition unit 10 may acquire the biological information Ig before the representation space 200 is presented, or may acquire the biological information Ig after the representation space 200 is presented. The biological information Ig may include at least one of heart rate information, perspiration amount information, or body temperature information of the subject 110. The biological information Ig of subject 110 may be acquired by a sensor provided in a wearable appliance worn by the subject 110.

The biological information Ig after the representation space 200 is presented may reflect a subconscious evaluation of the representation space 200 by the subject 110 who experienced the representation space 200. For example, when the subject 110 feels stress about the representation space 200, the subject 110 is more likely to become a state in which a sympathetic nerve is dominant over a parasympathetic nerve. When the sympathetic nerve is dominant over the parasympathetic nerve, a heart rate variability of the subject 110 is likely to be low, and a stressed state is likely to increase.

The state estimation unit 30 may estimate the state S based on the subject brain wave information Ib and the biological information Ig. The state estimation unit 30 may estimate the state S of the subject 110 based on the change from the first brain wave information Ib1 to the second brain wave information Ib2 and the biological information Ig.

A magnitude of a first power spectrum in a heart rate of the subject 110 is defined as LF, and a magnitude of a second power spectrum is defined as HF. A frequency band of the second power spectrum is a higher frequency band than a frequency band of the first power spectrum. The frequency band of the first power spectrum and the frequency band of the second power spectrum may not be overlapped. The frequency band of the first power spectrum is, for example, 0.04 to 0.15 Hz. The frequency band of the second power spectrum is, for example, 0.15 to 0.4 Hz.

A change from a proportion of the amplitude of the brain wave in a predetermined frequency band to the total amplitude As in the first brain wave information Ib1 to a proportion of the amplitude of the brain wave in the predetermined frequency band to the total amplitude As in the second brain wave information Ib2 is defined as change C. The state estimation unit 30 may estimate the state S based on the change C and a ratio of LF to HF, namely LF/HF.

As an example, when a proportion of a sum of the amplitude of the high beta wave and the amplitude of the gamma wave to the total amplitude As of the subject 110 after the representation space 200 is presented is greater than a proportion of that to the total amplitude As before the representation space 200 is presented, and the ratio of LF to HF, namely LF/HF, after the representation space 200 is presented is equal to or greater than a threshold, it may be assumed that an irritated state, a nervous state, or a stressed state of the subject 110 are increasing. When the ratio of LF to HF, namely LF/HF, is equal to or greater than the threshold, it may be determined that the subject 110 is in a state in which the sympathetic nerve is dominant over the parasympathetic nerve. When the ratio of LF to HF, namely LF/HF, is less than the threshold, it may be determined that the subject 110 is in a state in which the parasympathetic nerve is dominant over the sympathetic nerve. The threshold may be 2, 3, 4, or 5.

As an example, when the proportion of the sum of the amplitude of the high beta wave and the amplitude of the gamma wave to the total amplitude As of the subject 110 after the representation space 200 is presented is greater than the proportion of that to the total amplitude As before the representation space 200 is presented, and the ratio of LF to HF, namely LF/HF, after the representation space 200 is presented is less than the threshold, it may be assumed that an excited state of the subject 110 is increasing.

The state estimation unit 30 may estimate the state S based on a magnitude relationship between the ratio of LF to HF, namely LF/HF, after the representation space 200 is presented and the threshold for the ratio of LF to HF, and the change C. The threshold may be predetermined. When the proportion of the sum of the amplitude of the high beta wave and the amplitude of the gamma wave to the total amplitude As of the subject 110 after the representation space 200 is presented is greater than the proportion of that to the total amplitude As before the representation space 200 is presented, and the ratio of LF to HF, namely LF/HF, after the representation space 200 is presented is equal to or greater than the threshold, the state estimation unit 30 may estimate that the subject 110 is in a state S in which a sense of alertness toward the representation space 200 is increasing. When the proportion of the sum of the amplitude of the high beta wave and the amplitude of the gamma wave to the total amplitude As of the subject 110 after the representation space 200 is presented is greater than the proportion of that to the total amplitude As before the representation space 200 is presented, and the ratio of LF to HF, namely LF/HF, after the representation space 200 is provided is less than the threshold, the state estimation unit 30 may estimate that the subject 110 is in a state S in which a degree of excitement toward the representation space 200 is increasing.

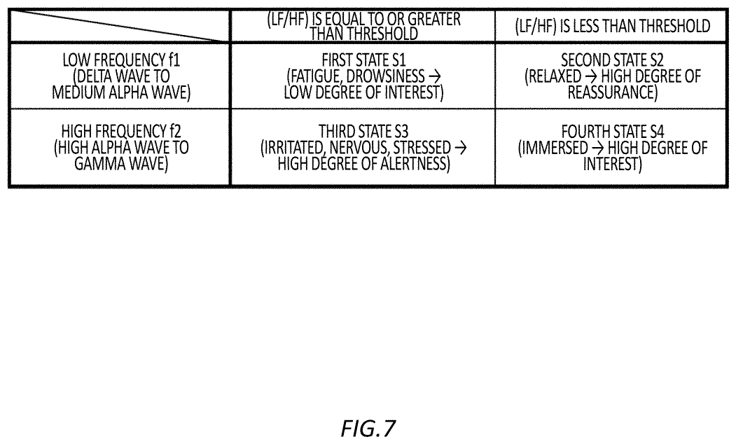

FIG. 7 is a diagram illustrating an example of a state S which is estimated by the state estimation unit 30. The state S may include a plurality of states of the subject 110, namely a first state S1 to an n-th state Sn. In the present example, the state S includes four states of the subject 110, namely the first state S1 to the fourth state S4. In FIG. 7, the brain wave at a low frequency f1 refers to at least one of the delta wave, the theta wave, the low alpha wave, or the medium alpha wave, and the brain wave at a high frequency f2 refers to at least one of the high alpha wave, the low beta wave, the high beta wave, or the gamma wave.

An amplitude of the brain wave of the subject 110 which is an amplitude of the brain wave in a predetermined frequency band is defined as an amplitude Af. The amplitude Af of the brain wave of the subject 110 before the representation space 200 is presented is defined as an amplitude Af1. The amplitude Af of the brain wave of the subject 110 after the representation space 200 is presented is defined as an amplitude Af2. The brain wave in the predetermined frequency band may be at least one of the low alpha wave, the medium alpha wave, the high alpha wave, the low beta wave, the high beta wave, the gamma wave, or the theta wave.

The state estimation unit 30 may estimate the state S based on a change from a proportion of the amplitude Af1 to the total amplitude As to a proportion of the amplitude Af2 to the total amplitude As, and the ratio of LF to HF, namely LF/HF. The state S may be one state S of the plurality of states S of the subject 110, namely any one of the first state S1 to the n-th state Sn.

In the present example, the first state S1 is a state of the subject 110 when, in the brain wave at the low frequency f1, the proportion of the amplitude Af2 to the total amplitude As is greater than the proportion of the amplitude Af1 to the total amplitude As, and the ratio of LF to HF, namely LF/HF, after the representation space 200 is presented is equal to or greater than the threshold. When the subject 110 is in the first state S1, it may be assumed that a fatigued state and a drowsy state of the subject 110 are increasing. When the subject 110 is in the first state S1, the state estimation unit 30 may estimate that a degree of interest of the subject 110 toward the representation space 200 is decreasing.

In the present example, the second state S2 is a state of the subject 110 when, in the brain wave at the low frequency f1, the proportion of the amplitude Af2 to the total amplitude As is greater than the proportion of the amplitude Af1 to the total amplitude As, and the ratio of LF to HF, namely LF/HF, after the representation space 200 is presented is less than the threshold. When the subject 110 is in the second state S2, it may be assumed that a relaxed state of the subject 110 is increasing. When the subject 110 is in the second state S2, the state estimation unit 30 may estimate that a degree of reassurance of the subject 110 toward the representation space 200 is increasing.

In the present example, the third state S3 is a state of the subject 110 when, in the brain wave at the high frequency f2, the proportion of the amplitude Af2 to the total amplitude As is greater than the proportion of the amplitude Af1 to the total amplitude As, and the ratio of LF to HF, namely LF/HF, after the representation space is presented is equal to or greater than the threshold. When the subject 110 is in the third state S3, it may be assumed that the irritated state, the nervous state, or the stressed state of the subject 110 are increasing. When the subject 110 is in the third state S3, the state estimation unit 30 may estimate that a degree of alertness of the subject 110 toward the representation space 200 is increasing.

In the present example, the fourth state S4 is a state of the subject 110 when, in the brain wave at the high frequency f2, the proportion of the amplitude Af2 to the total amplitude As is greater than the proportion of the amplitude Af1 to the total amplitude As, and the ratio of LF to HF, namely LF/HF, after the representation space 200 is presented is less than the threshold. When the subject 110 is in the fourth state S4, it may be assumed that the immersed state of the subject 110 is increasing. When the subject 110 is in the fourth state S4, the state estimation unit 30 may estimate that the degree of interest of the subject 110 toward the representation space 200 is increasing.

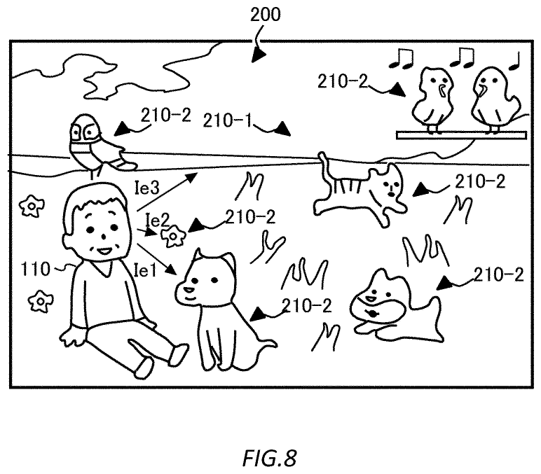

FIG. 8 is a diagram illustrating an example of line-of-sight information of the subject 110. In FIG. 8, the line-of-sight information is illustrated together with the representation space 200 and the subject 110 in FIG. 4. The line-of-sight information is defined as line-of-sight information le. The line-of-sight information le is information which indicates a position visually recognized by the subject 110 in the representation space 200. The line-of-sight information le may be acquired from an image of eyes of the subject 110. The image of the eyes may be acquired by an imaging device provided in a wearable appliance worn by the subject 110, or may be acquired by an imaging device provided in another appliance.

The information acquisition unit 10 may acquire the line-of-sight information le. The information acquisition unit 10 may acquire a type of the image element 210 of interest to which the subject 110 is paying attention, based on the line-of-sight information le. The type of the image element 210 is defined as type K′. The information acquisition unit 10 may acquire the type K′ of an image element 210 based on the line-of-sight information le indicating the position visually recognized by the subject 110, and the image element 210 presented at the position by the space presentation unit 20. The type K′ may refer to the type of scenery such as a sea, a mountain, and a sky, or may refer to the type of a living body such as an animal and a plant. In the example of FIG. 8, an image element 210-1 is scenery, and an image element 210-2 is a living body. In FIG. 8, the line-of-sight information Ie1 is the line-of-sight information le when the subject 110 visually recognizes a dog as the image element 210-2, the line-of-sight information Ie2 is the line-of-sight information le when the subject 110 visually recognizes a flower as the image element 210-2, and the line-of-sight information Ie3 is the line-of-sight information le when the subject 110 visually recognizes scenery as the image element 210-1.

The space presentation unit 20 may adjust the representation space 200 based on the line-of-sight information Ie. The space presentation unit 20 may adjust the representation space 200 based on the type K′ of the image element 210 acquired by the information acquisition unit 10. In the example of FIG. 8, the dog presented in the representation space 200 may be the image element 210 which may put the state S of the subject 110 into the desired state S. In such a case, the space presentation unit 20 may adjust the representation space 200 to display still another dog in the representation space 200. Accordingly, the subject 110 is more likely to become the desired state S.

The space presentation unit 20 may adjust the representation space 200 based on the change from the first brain wave information Ib1 to the second brain wave information Ib2, and the line-of-sight information le. The space presentation unit 20 may adjust the representation space 200 based on the change from the first brain wave information Ib1 to the second brain wave information Ib2, and the type K′ of the image element 210 based on the line-of-sight information le. Based on the change from the first brain wave information Ib1 to the second brain wave information Ib2, and the type K′ of the image element 210 based on the line-of-sight information le, the space presentation unit 20 may adjust the representation space 200 to present the image element 210 of different type K′ than the image element 210 based on the line-of-sight information le in the representation space 200, or may adjust the representation space 200 to present the image element 210 of a same type K′ as the image element 210 based on the line-of-sight information le in the representation space 200.

For example, when the image element 210-2 based on the line-of-sight information Ie1 is a dog, and the proportion of the amplitude of the theta wave to the total amplitude As in the second brain wave information Ib2 is increased from the proportion of the amplitude of the theta wave to the total amplitude As in the first brain wave information Ib1, there is a high probability that the fatigue and the drowsiness of the subject 110 are not relieved by the image element 210-2, namely the dog in the present example. In such a case, the space presentation unit 20 may present the image element 210-1 such as a sky in the representation space 200, instead of the image element 210-2, namely the dog in the present example.

For example, when the image element 210 based on the line-of-sight information Ie1 is a dog, and the proportion of the sum of the amplitude of the low alpha wave and the amplitude of the medium alpha wave to the total amplitude As in the second brain wave information Ib2 is increased from the proportion of the sum of the amplitude of the low alpha wave and the amplitude of the medium alpha wave to the total amplitude As in the first brain wave information Ib1, there is a high probability that the degree of relaxation of the subject 110 is enhanced by the image element 210-2, namely the dog in the present example. In such a case, in addition to one image element 210-2, namely the dog in the present example, the space presentation unit 20 may present another image element 210-2 such as another dog in the representation space 200. For example, when the image element 210 based on the line-of-sight information Ie2 is a flower, and the proportion of the sum of the amplitude of the high alpha wave and the amplitude of the low beta wave to the total amplitude As in the second brain wave information Ib2 is increased from the proportion of the sum of the amplitude of the high alpha wave and the amplitude of the low beta wave to the total amplitude As in the first brain wave information Ib1, there is a high probability that the degree of immersion of the subject 110 is enhanced by the image element 210-2, namely the flower in the present example. In such a case, in addition to one image element 210-2, namely the flower in the present example, the space presentation unit 20 may present another image element 210-2 such as another flower in the representation space 200.

A period for which the representation space 200 is presented to the subject 110 is defined as a period Te. The period Te may be time from a moment at which the presentation of the representation space 200 to the subject 110 is started to a moment at which the presentation of the representation space 200 is finished. In the example of FIG. 1 and FIG. 8, the period Te may be the time from a moment at which the subject 110 enters another room where the representation space 200 is presented to a moment at which the subject 110 leaves the another room.

The information acquisition unit 10 may acquire time for which the subject 110 visually recognizes one image element 210, based on the line-of-sight information le. The time is defined as time Ts. The time Ts may refer to time for which the subject 110 continues to visually recognize the one image element 210. When the subject 110 visually recognizes one image element 210, then visually recognizes another image element 210, and then visually recognizes the one image element 210 again during the period Te, the time Ts may be a sum of the time for which the subject 110 continues to visually recognize the one image element 210.

The space presentation unit 20 may adjust the representation space 200 based on a proportion of the time Ts to the period Te. The space presentation unit 20 may adjust the representation space 200 based on a magnitude relationship between the proportion of the time Ts to the period Te and a threshold for the proportion of the time Ts to the period Te. The threshold is defined as a threshold Tth1.

When the proportion of the time Ts to the period Te is greater than the threshold Tth1, the space presentation unit 20 may present an image element 210 of a same type K′ as the one image element 210 in the representation space 200. When the proportion of the time Ts to the period Te is greater than the threshold Tth1, there is a high probability that the one image element 210 is an image element 210 which may put the state S of the subject 110 into the desired state S.

Accordingly, the space presentation unit 20 may present the image element 210 of the same type K′ as the one image element 210 in the representation space 200. When the proportion of the time Ts to the period Te is less than or equal to the threshold Tth1, the space presentation unit 20 may present an image element 210 of a different type K′ than the one image element 210 in the representation space 200. When the proportion of the time Ts to the period Te is less than or equal to the threshold Tth1, there is a high probability that the one image element 210 is not an image element 210 which may put the state S of the subject 110 into the desired state S. Accordingly, the space presentation unit 20 may present an image element 210 of a different type K′ than the one image element 210 in the representation space 200.

The information acquisition unit 10 may generate a question to be presented to the subject 110 based on the first brain wave information Ib1. The information acquisition unit 10 may determine a type of the question to be presented to the subject 110 and generate a question of the determined type, based on the first brain wave information Ib1. For example, when the proportion of the sum of the amplitude of the low alpha wave and the amplitude of the medium alpha wave to the total amplitude As in the first brain wave information Ib1 is increasing over time, the information acquisition unit 10 may generate a question for enhancing the degree of relaxation of the subject 110. The question for enhancing the degree of relaxation is, for example, a question such as asking a favorite activity of the subject 110 such as, for example, mountain climbing, swimming in the sea or the like.

For example, when the proportion of the sum of the amplitude of the high alpha wave and the amplitude of the low beta wave to the total amplitude As in the first brain wave information Ib1 is increasing over time, the information acquisition unit 10 may generate a question for enhancing the degree of immersion of the subject 110. The question for enhancing the degree of immersion is, for example, a question such as asking a favorite animal, a favorite place or the like of the subject.

The space presentation unit 20 may present the question generated by the information acquisition unit 10 in the representation space 200. The space presentation unit 20 may present the representation space 200 based on an answer of the subject 110 to the question. Accordingly, the subject 110 is more likely to become the desired state S.

The information acquisition unit 10 may further generate another question to be presented to the subject 110 based on the change from the first brain wave information Ib1 to the second brain wave information Ib2. The state S of the subject 110 may not become the desired state S, although the space presentation unit 20 has adjusted the representation space 200 based on the answer to the question generated by the information acquisition unit 10. In such a case, the information acquisition unit 10 may further generate another question to be presented to the subject 110. The space presentation unit 20 may adjust the representation space 200 based on the answer to the another question of the subject 110. The information acquisition unit 10 may repeat the generation of a question until the state S of the subject 110 becomes the desired state S.

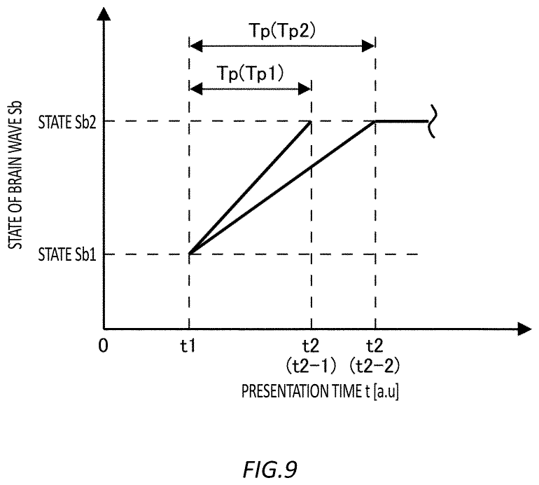

FIG. 9 is a diagram illustrating an example of a relationship between presentation time t of the representation space 200 and a state of the brain wave of the subject 110. In FIG. 9, the presentation time of the representation space 200 is shown as time t. The state of the brain wave of the subject 110 is defined as a state Sb. The state Sb may refer to a proportion of the amplitude of a predetermined frequency band to the total amplitude As at one timing t, or may refer to a ratio between the amplitude of one frequency band such as the alpha wave and the amplitude of another frequency band such as the beta wave at one timing t.

In the example of FIG. 9, the brain wave of the subject 110 is in a state Sb1 at a first timing t1, and in a state Sb2 at a second timing t2. The second timing t2 is a timing of a change from the first brain wave information Ib1 to the second brain wave information Ib2. When the subject brain wave information Ib changes from the first brain wave information Ib1 to the desired second brain wave information Ib2, the timing of the change from the first brain wave information Ib1 to the second brain wave information Ib2 may be a timing at which the change to the second brain wave information Ib2 is finished. In the example of FIG. 9, there are a plurality of second timings t2. A second timing t2-1 is before a second timing t2-2. In the example of FIG. 9, assume that the space presentation unit 20 presents the representation space 200 depending on the first brain wave information Ib1 at the first timing t1.

The time from the first timing t1 to the second timing t2 is defined as time Tp. The time from the first timing t1 to the second timing t2-1 is defined as time Tp1, and the time from the first timing t1 to the second timing t2-2 is defined as time Tp2. In the present example, the time Tp1 is shorter than the time Tp2.

The information acquisition unit 10 may acquire the first timing t1 and the second timing t2. The space presentation unit 20 may adjust the representation space 200 based on a relationship between the first timing t1 and the second timing t2.

The representation space 200 depending on the first brain wave information Ib1 may change over time. From the first timing t1 to the second timing t2, the space presentation unit 20 may present the representation space 200 which changes over time. The representation space 200 which changes over time may refer to the representation space 200 where a position of the image element 210 changes with time t. The information acquisition unit 10 may acquire the type K of the representation space 200 or the type K′ of the image element 210 based on a change of the representation space 200 over time. The information acquisition unit 10 may acquire the type K of the representation space 200 or the type K′ of the image element 210 based on a moving speed of the image element 210 in the representation space 200. A threshold for the moving speed is defined as a threshold Vth.

The information acquisition unit 10 may acquire the type K of the representation space 200 or the type K′ of the image element 210 based on a magnitude relationship between the moving speed of the image element 210 and the threshold Vth. When the moving speed of the image element 210 is less than or equal to the threshold Vth, the information acquisition unit 10 may acquire information indicating that the type K of the representation space 200 is one type K1, or acquire information indicating that the type K′ of the image element 210 is one type K1′. When the moving speed of the image element 210 is greater than the threshold Vth, the information acquisition unit 10 may acquire information indicating that the type K of the representation space 200 is another type K2 which is different from the one type K1, or acquire information indicating that the type K′ of the image element 210 is another type K2′ which is different from the one type K1′.

The moving speed of the image element 210 being less than or equal to the threshold Vth may refer to, for example, when the image element 210 is scenery such as a sea and a mountain, a relative position of the subject 110 and the image element 210 changing slowly with time t, such as when the subject 110 is enjoying the scenery from inside a moving train. The moving speed of the image element 210 being greater than the threshold Vth may refer to, for example, when the image element 210 is a living body such as a dog and a cat, the relative position of the subject 110 and the image element 210 changing rapidly with time t, such as when a dog, a cat or the like suddenly jumped out from a bush in a grassland in the representation space 200. The moving speed of the image element 210 being greater than the threshold Vth may refer to, for example, when the representation space 200 is unexplored land and the subject 110 is experiencing an activity such as taking an adventure in the unexplored land, the relative position of the subject 110 and the image element 210 changing rapidly with time t, such as when a beast suddenly jumped out from behind rocks in the representation space 200.

The space presentation unit 20 may adjust the representation space 200 based on the type K of the representation space 200 or the type K′ of the image element 210. When the type K of the representation space 200 is the one type K1 described above, for example, the state S of the subject 110 may change gradually and then become the desired state S, such as when the subject 110 continues to enjoy the scenery from inside the moving train. Accordingly, the second brain wave information Ib2 after the time Tp, namely the time Tp2 in the present example, has passed may be the subject brain wave information Ib when the subject 110 is in the desired state S. Accordingly, the space presentation unit 20 may present the representation space 200 of the one type K1 even after the time Tp has passed. A same applies when the type K′ of the image element 210 is the one type K1′ described above.

When the type K of the representation space 200 is the another type K2 described above, for example, the state S of the subject 110 may change rapidly, such as when the subject 110 sees a dog, a cat or the like suddenly jump out from a bush of a grassland in the representation space 200 so that the state S of the subject 110 changes rapidly. Accordingly, the second brain wave information Ib2 after the time Tp, namely the time Tp1 in the present example has passed may be different from the subject brain wave information Ib when the subject 110 is in the desired state S. Accordingly, after the time Tp has passed, the space presentation unit 20 may not present the representation space 200 of the another type K2, or may present the representation space 200 of a different type K from the another type K2. A same applies when the type K′ of the image element 210 is the another type K2′ described above.

The information acquisition unit 10 may acquire the second brain wave information Ib2 at a timing based on the type K of the representation space 200 or the type K′ of the image element 210. The information acquisition unit 10 may change the second timing t2 at which it acquires the second brain wave information Ib2 for each type K of the representation space 200. The information acquisition unit 10 may change the second timing t2 at which it acquires the second brain wave information Ib2 for each type K′ of the image element 210. The information acquisition unit 10 becomes more likely to acquire the second brain wave information Ib2 after the change from the first brain wave information Ib1 to the second brain wave information Ib2 has finished, since the information acquisition unit 10 acquires the second brain wave information Ib2 at a timing based on the type K of the representation space 200 or the type K′ of the image element 210.

For example, when the type K of the representation space 200 is the one type K1 described above, the second brain wave information Ib2 may change slowly with a movement of the image element 210. For example, when the type K of the representation space 200 is the another type K2 described above, the second brain wave information Ib2 may change more rapidly with the movement of the image element 210 than in the case of the one type K1. Accordingly, the second timing t2 when the type K of the representation space 200 is the one type K1 described above tends to become later than the second timing t2 when the type K of the representation space 200 is the another type K2. In the present example, the information acquisition unit 10 assumes that the second timing t2 at which it acquires the second brain wave information Ib2 when the type K of the representation space 200 is the one type K1 described above is later than the second timing t2 at which it acquires the second brain wave information Ib2 when the type K of the representation space 200 is the another type K2. Accordingly, the information acquisition unit 10 becomes more likely to acquire the second brain wave information Ib2 after the change from the first brain wave information Ib1 to the second brain wave information Ib2 has finished.

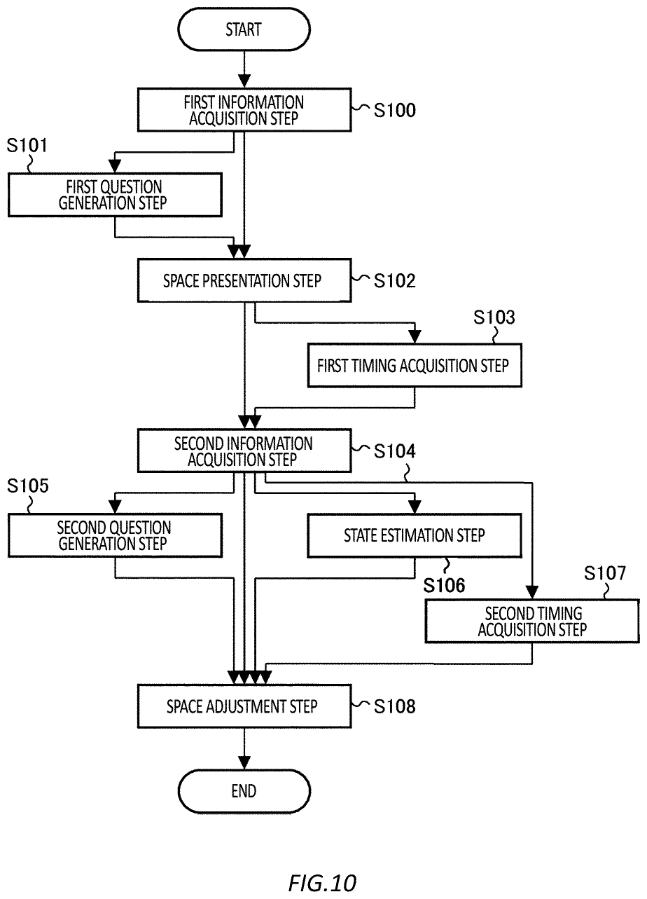

FIG. 10 is a flowchart illustrating an example of a space presentation method according to an embodiment of the present invention. The space presentation method includes a first information acquisition step S100, a space presentation step S102, a second information acquisition step S104, and a space adjustment step S108. The space presentation method may include a state estimation step S106, a first question generation step S101, a second question generation step S105, a first timing acquisition step S103, and a second timing acquisition step S107. The space presentation method according to an embodiment of the present invention will be described using the space presentation apparatus 100 illustrated in FIG. 5 as an example.

The first information acquisition step S100 is a step in which the information acquisition unit 10 acquires the first brain wave information Ib1 before the representation space 200 is presented. The space presentation step S102 is a step in which the space presentation unit 20 presents the representation space 200 depending on the first brain wave information Ib1. The second information acquisition step S104 is a step in which the information acquisition unit 10 acquires the second brain wave information Ib2 after the representation space 200 is presented. The space adjustment step S108 is a step in which the space presentation unit 20 adjusts the representation space 200 based on the change from the first brain wave information Ib1 to the second brain wave information Ib2.

The state estimation step S106 is a step in which the state estimation unit 30 estimates the state S of the subject 110 based on the subject brain wave information Ib of the subject 110. The state estimation step S106 may be a step in which the state estimation unit 30 estimates the state S of the subject 110 based on the change from the first brain wave information Ib1 to the second brain wave information Ib2. The space adjustment step S108 may be a step in which the space presentation unit 20 adjusts the representation space 200 based on the state S of the subject 110.

The first information acquisition step S100 or the second information acquisition step S104 may be a step in which the information acquisition unit 10 further acquires the biological information Ig of the subject 110. The state estimation step S106 may be a step in which the state estimation unit 30 estimates the state S of the subject 110 based on the subject brain wave information Ib and the biological information Ig of the subject 110. The state estimation step S106 may be a step in which the state estimation unit 30 estimates the state S of the subject 110 based on the change from the first brain wave information Ib1 to the second brain wave information Ib2 and the biological information Ig.

The state estimation step S106 may be a step in which the state estimation unit 30 estimates the state S based on the change from the proportion of the amplitude of the brain wave in a predetermined frequency band Af to the total amplitude As in the first brain wave information Ib1 to the proportion of the amplitude of the brain wave in the predetermined frequency band Af to the total amplitude As in the second brain wave information Ib2, and the ratio of the first power spectrum LF to the second power spectrum HF, namely LF/HF, in the heart rate of the subject 110.

The state estimation step S106 may be a step in which the state estimation unit 30 estimates the state S based on the magnitude relationship between the ratio of LF to HF, namely LF/HF, after the representation space 200 is presented and the predetermined threshold for the ratio of LF to HF, namely LF/HF, and the change from the proportion of the amplitude of a brain wave to the total amplitude As in the first brain wave information Ib1 to the proportion of the amplitude of the brain wave to the total amplitude As in the second brain wave information Ib2.

The state S may include a plurality of states of the subject 110. The state estimation step S106 may be a step in which the state estimation unit 30 estimates one state S among the plurality of states S, based on the change from the proportion of the amplitude of the brain wave in a predetermined frequency band Af to the total amplitude As in the first brain wave information Ib1 to the proportion of the amplitude of the brain wave in the predetermined frequency band Af to the total amplitude As in the second brain wave information Ib2, and the ratio of LF to HF, namely LF/HF.

The first question generation step S101 is a step in which the information acquisition unit 10 generates a question to be presented to the subject 110 based on the first brain wave information Ib1. The space presentation step S102 may be a step in which the space presentation unit 20 presents the representation space 200 based on the answer to the question. The second question generation step S105 is a step in which the information acquisition unit 10 further generates another question to be presented to the subject 110 based on the change from the first brain wave information Ib1 to the second brain wave information Ib2. The space adjustment step S108 may be a step in which the space presentation unit 20 adjusts the representation space 200 based on the answer of the subject 110 to the another question.

The second information acquisition step S104 may be a step in which the information acquisition unit 10 further acquires the line-of-sight information le indicating the position visually recognized by the subject 110. The space adjustment step S108 may be a step in which the space presentation unit 20 adjusts the representation space 200 based on the line-of-sight information le. The space adjustment step S108 may be a step in which the space presentation unit 20 adjusts the representation space 200 based on the change from the first brain wave information Ib1 to the second brain wave information Ib2 and the line-of-sight information Ie.

The first timing acquisition step S103 is a step in which the information acquisition unit 10 acquires the first timing t1 at which the space presentation unit 20 presents the representation space 200 depending on the first brain wave information Ib1 in the space presentation step S102. The second timing acquisition step S107 is a step in which the information acquisition unit 10 acquires the second timing t2 at which the first brain wave information Ib1 changes to the second brain wave information Ib2. The space adjustment step S108 is a step in which the space presentation unit 20 adjusts the representation space 200 based on the relationship between the first timing t1 and the second timing t2.

The space presentation step S102 may be a step in which the space presentation unit 20 presents the representation space 200 which changes over time, depending on the first brain wave information Ib1, from the first timing t1 to the second timing t2. The second information acquisition step S104 may be a step in which the information acquisition unit 10 acquires the type K of the representation space 200 based on the change of the representation space 200 over time. The space adjustment step S108 may be a step in which the space presentation unit 20 adjusts the representation space 200 based on the type K of the representation space 200. The second information acquisition step S104 may be a step in which the information acquisition unit 10 acquires the second brain wave information Ib2 at a timing based on the type K of the representation space 200.

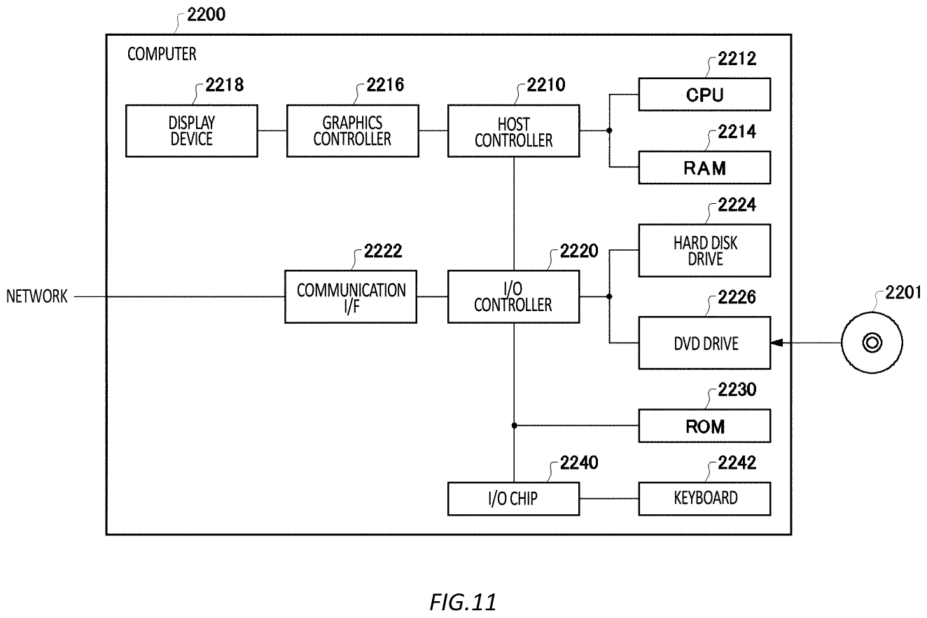

FIG. 11 is a diagram illustrating an example of a computer 2200 in which the space presentation apparatus 100 according to an embodiment of the present invention may be embodied entirely or partially. A program installed on the computer 2200 may cause the computer 2200 to function as operations associated with the space presentation apparatus 100 according to an embodiment of the present invention or one or more sections of the space presentation apparatus 100, or may cause the computer 2200 to execute the operations or the one or more sections, or may cause the computer 2200 to execute each step according to the method of the present invention (see FIG. 10). The program may be executed by a CPU 2212 to cause the computer 2200 to execute certain operations associated with some or all of the blocks in the flowchart in FIG. 10 and the block diagram in FIG. 5 described herein.

The computer 2200 according to an embodiment of the present invention includes the CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes input/output units such as a communication interface 2222, a hard disk drive 2224, a DVD-ROM drive 2226, and an IC card drive. The communication interface 2222, the hard disk drive 2224, the DVD-ROM drive 2226, the IC card drive, and the like are connected to the host controller 2210 via an input/output controller 2220. The computer further includes legacy input/output units such as a ROM 2230 and a keyboard 2242. The ROM 2230, the keyboard 2242, and the like are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 on a frame buffer or the like provided in the RAM 2214 or in the RAM 2214 itself to cause the image data to be displayed on the display device 2218.

The communication interface 2222 communicates with another electronic device via a network. The hard disk drive 2224 stores programs and data used by the CPU 2212 in the computer 2200. The DVD-ROM drive 2226 reads the programs or the data from the DVD-ROM 2201, and provides the read programs or data to the hard disk drive 2224 via the RAM 2214. The IC card drive reads programs and data from an IC card, or writes programs and data to the IC card. The ROM 2230 stores a boot program or the like executed by the computer 2200 at a time of activation, or a program which depends on hardware of the computer 2200. The input/output chip 2240 may connect various input/output units via a parallel port, a serial port, a keyboard port, a mouse port, or the like to the input/output controller 2220.

Programs are provided by a computer-readable medium such as the DVD-ROM 2201 or the IC card. The programs are read from the computer-readable medium, are installed in the hard disk drive 2224, the RAM 2214, or the ROM 2230 which is also an example of the computer-readable medium, and are executed by the CPU 2212. The information processing described in these programs is read by the computer 2200, and provides cooperation between the programs and the various types of hardware resources described above. An apparatus or method may be constructed by realizing the operation or processing of information in accordance with the usage of the computer 2200.

For example, when communication is executed between the computer 2200 and an external device, the CPU 2212 may execute a communication program loaded onto the RAM 2214 to instruct the communication interface 2222 to perform communication processing, based on the processing described in the communication program. The communication interface 2222, under control of the CPU 2212, reads transmission data stored in a transmission buffering region provided in a recording medium such as the RAM 2214, the hard disk drive 2224, the DVD-ROM 2201, or the IC card, and transmits the read transmission data to a network or writes reception data received from a network in a reception buffering region or the like provided on the recording medium.

The CPU 2212 may cause all or a necessary portion of a file or a database stored in an external recording medium such as the hard disk drive 2224, the DVD-ROM drive 2226 (DVD-ROM 2201), the IC card, or the like to be read by the RAM 2214. The CPU 2212 may execute various types of processing on the data on the RAM 2214. The CPU 2212 may then write the processed data back to the external recording medium.

Various types of information, such as various types of programs, data, tables, and databases, may be stored in the recording medium to undergo information processing. The CPU 2212 may execute various types of processing described in the present disclosure on the data read from the RAM 2214, which include various types of operations, information processing, conditional judgment, conditional branch, unconditional branch, search or replacement of information, or the like and are specified by an instruction sequence of programs. The CPU 2212 may write the result back to the RAM 2214.

The CPU 2212 may search for information in a file, a database, or the like in the recording medium. For example, when a plurality of entries, each having an attribute value of a first attribute associated with an attribute value of a second attribute, are stored in the recording medium, the CPU 2212 may search for an entry which matches the condition whose attribute value of the first attribute is specified, from among the plurality of entries, read the attribute value of the second attribute stored in the entry, and read the second attribute value to acquire the attribute value of the second attribute associated with the first attribute satisfying the predetermined condition.

The programs or software modules described above may be stored in the computer-readable medium on the computer 2200 or of the computer 2200. A recording medium such as a hard disk or a RAM provided in a server system connected to a dedicated communication network or the Internet may be used as the computer-readable medium. The programs may be provided to the computer 2200 by the recording medium.

While the present invention has been described above with the embodiments, the technical scope of the present invention is not limited to the scope of the above-described embodiments. It is apparent to persons skilled in the art that various alterations or improvements may be made to the above-described embodiments. It is apparent from the description of the claims that the embodiments in which such alterations or improvements are made may also be included in the technical scope of the present invention.

It should be noted that each process such as an operation, a procedure, a step, a stage, or the like in an apparatus, a system, a program, and a method shown in the claims, the specification and the drawings may be realized in any order as long as the order is not explicitly specified by “prior to,” “before,” or the like particularly and as long as an output from a previous process is not used in a later process. Even if an operational flow in the claims, the specification, and the drawings is described using phrases such as “first” and “then” for convenience, it does not necessarily mean that it must be performed in this order.

### EXPLANATION OF REFERENCES

10: information acquisition unit; 20: space presentation unit; 30: state estimation unit; 40: storage unit; 90: control unit; 100: space presentation apparatus; 110: subject; 200: representation space; 210: image element; 2200: computer; 2201: DVD-ROM; 2210: host controller; 2212: CPU; 2214: RAM; 2216: graphics controller; 2218: display device; 2220: input/output controller; 2222: communication interface; 2224: hard disk drive; 2226: DVD-ROM drive; 2230: ROM; 2240: input/output chip; 2242: keyboard.

What is claimed is:

1. A space presentation apparatus comprising:
an information acquisition unit which acquires subject brain wave information which is brain wave information of a subject; and
a space presentation unit which presents a representation space to the subject, wherein the information acquisition unit acquires first brain wave information which is the subject brain wave information before the representation space is presented,
the space presentation unit presents the representation space depending on the first brain wave information,
the information acquisition unit acquires second brain wave information which is the subject brain wave information after the representation space is presented, and
the space presentation unit adjusts the representation space based on a change from the first brain wave information to the second brain wave information.

2. The space presentation apparatus according to claim 1, further comprising:
a state estimation unit which estimates a state of the subject based on the subject brain wave information, wherein
the space presentation unit presents the representation space based on the state of the subject.

3. The space presentation apparatus according to claim 2, wherein
the information acquisition unit further acquires biological information of the subject, and
the state estimation unit estimates a state of the subject based on the subject brain wave information and the biological information.

4. The space presentation apparatus according to claim 3, wherein
the state estimation unit estimates the state based on a change from a proportion of an amplitude of a brain wave in a predetermined frequency band to a total amplitude in the first brain wave information to the proportion of the amplitude of the brain wave in the frequency band to the total amplitude in the second brain wave information, and a ratio of a magnitude of a first power spectrum to a magnitude of a second power spectrum in a heart rate of the subject,
the total amplitude is a sum of amplitudes of an alpha wave, a beta wave, a theta wave, a gamma wave, and a delta wave, and
a frequency band of the second power spectrum is a higher frequency band than a frequency band of the first power spectrum.

5. The space presentation apparatus according to claim 4, wherein
the state estimation unit estimates the state based on a magnitude relationship between the ratio of the magnitude of the first power spectrum to the magnitude of the second power spectrum after the representation space is presented and a predetermined threshold for the ratio of the magnitude of the first power spectrum to the magnitude of the second power spectrum, and the change from the proportion of the amplitude of the brain wave to the total amplitude in the first brain wave information to the proportion of the amplitude of the brain wave to the total amplitude in the second brain wave information.

6. The space presentation apparatus according to claim 4, wherein
the state includes a plurality of states of the subject, and
the state estimation unit estimates the state which is one of the plurality of states, based on the change from the proportion of the amplitude of the brain wave to the total amplitude in the first brain wave information to the proportion of the amplitude of the brain wave to the total amplitude in the second brain wave information, and the ratio of the magnitude of the first power spectrum to the magnitude of the second power spectrum.

7. The space presentation apparatus according to claim 6, wherein the brain wave in the frequency band is at least one of the delta wave, the theta wave, a low alpha wave, or a medium alpha wave.

8. The space presentation apparatus according to claim 6, wherein the brain wave in the frequency band is at least one of a high alpha wave, a low beta wave, a high beta wave, or the gamma wave.

9. The space presentation apparatus according to claim 1, wherein
the information acquisition unit generates a question to be presented to the subject based on the first brain wave information, and
the space presentation unit presents the representation space based on an answer to the question.

10. The space presentation apparatus according to claim 2, wherein
the information acquisition unit generates a question to be presented to the subject based on the first brain wave information, and
the space presentation unit presents the representation space based on an answer to the question.

11. The space presentation apparatus according to claim 3, wherein
the information acquisition unit generates a question to be presented to the subject based on the first brain wave information, and
the space presentation unit presents the representation space based on an answer to the question.

12. The space presentation apparatus according to claim 1, wherein
the information acquisition unit further acquires line-of-sight information which indicates a position visually recognized by the subject, and
the space presentation unit adjusts the representation space based on the line-of-sight information.

13. The space presentation apparatus according to claim 2, wherein
the information acquisition unit further acquires line-of-sight information which indicates a position visually recognized by the subject, and
the space presentation unit adjusts the representation space based on the line-of-sight information.

14. The space presentation apparatus according to claim 3, wherein
the information acquisition unit further acquires line-of-sight information which indicates a position visually recognized by the subject, and
the space presentation unit adjusts the representation space based on the line-of-sight information.

15. The space presentation apparatus according to claim 1, wherein
the information acquisition unit further acquires a first timing at which the space presentation unit presents the representation space depending on the first brain wave information, and a second timing at which the first brain wave information changes to the second brain wave information, and
the space presentation unit adjusts the representation space based on a relationship between the first timing and the second timing.

16. The space presentation apparatus according to claim 2, wherein
the information acquisition unit further acquires a first timing at which the space presentation unit presents the representation space depending on the first brain wave information, and a second timing at which the first brain wave information changes to the second brain wave information, and
the space presentation unit adjusts the representation space based on a relationship between the first timing and the second timing.

17. The space presentation apparatus according to claim 15, wherein
the space presentation unit presents the representation space which changes over time, depending on the first brain wave information, from the first timing to the second timing,
the information acquisition unit acquires a type of the representation space based on a change of the representation space over time, and
the space presentation unit adjusts the representation space based on the type of the representation space.

18. The space presentation apparatus according to claim 15, wherein the information acquisition unit acquires the second brain wave information at a timing based on a type of the representation space.

19. A space presentation method comprising:
acquiring, by an information acquisition unit, first information by acquiring first brain wave information which is brain wave information of a subject before a representation space is presented;
presenting, by a space presentation unit, a space by presenting the representation space depending on the first brain wave information;
acquiring, by the information acquisition unit, second information by acquiring second brain wave information which is the brain wave information of the subject after the representation space is presented; and
adjusting, by the space presentation unit, the space by adjusting the representation space based on a change from the first brain wave information to the second brain wave information.

20. A non-transitory computer-readable medium having recorded thereon a space presentation program which, when executed by a computer, causes the computer to perform operations comprising:
acquiring first information by acquiring first brain wave information which is brain wave information of a subject before a representation space is presented;
presenting a space by presenting the representation space depending on the first brain wave information;
acquiring second information by acquiring second brain wave information which is the brain wave information of the subject after the representation space is presented; and
adjusting the space by adjusting the representation space based on a change from the first brain wave information to the second brain wave information.
